(19) United States
(12) Patent Application Publication
(10) Pub. No.: US 2015/0078513 A1
(43) Pub. Date: Mar. 19, 2015

(54) DENTAL X-RAY IMAGING SYSTEM HAVING HIGHER SPATIAL RESOLUTION

(71) Applicant: Seung H. BAEK, La Habra, CA (US)

(72) Inventor: Seung H. BAEK, La Habra, CA (US)

(21) Appl. No.: 14/485,582

(22) Filed: Sep. 12, 2014

Related U.S. Application Data
(60) Provisional application No. 61/877,850, filed on Sep. 13, 2013.
(60) Provisional application No. 62/000,358, filed on May 19, 2014.

(51) Int. Cl. A61B 6/14; A61B 6/00; A61B 5/00; A61B 6/03

(52) U.S. Cl. CPC A61B 6/14; A61B 6/032; A61B 6/4085; A61B 6/4241; A61B 6/466; A61B 6/5217; A61B 5/4547; A61B 5/4552
USPC 378/19

(57) ABSTRACT

A dental cone beam computed tomography (CBCT) system, with a photon generator configured to emit x-ray photons; a photon detector spaced apart from the photon generator so as to accommodate at least a portion of a human mouth therebetween, the photon detector configured to receive the x-ray photons; and a processor in electronic communication with the photon detector. The photon detector is a direct-conversion detector configured to convert each received x-ray photon directly to a corresponding electrical signal, to determine information corresponding to a spatial pattern of the electrical signals, and to transmit the information to the processor. The processor is further configured to generate an image of the portion of the human mouth from the transmitted information.

This application claims priority to, and the benefit of, U.S. Provisional Application No. 61/877,850 filed on Sep. 13, 2013, and U.S. Provisional Application No. 62/000,358 filed on May 19, 2014, the contents of each of which are hereby incorporated by reference in their entireties.

### BRIEF DESCRIPTION

This invention relates generally to dental imaging. More specifically, this invention relates to dental x-ray imaging systems having higher spatial resolution.

### BACKGROUND

Current dental cone beam computed tomography (CBCT) systems are built upon image intensifiers or indirect detection using scintillators (CsI or Gadox). A typical dental CBCT system uses 200-500 frames of 2D images for reconstruction of 3D images, for which spatial resolution ranges from about 1 to about 7 line pairs/mm. However, 3D models constructed using the image intensifiers or indirect detection methods of conventional 2D CBCT images are not dimensionally accurate enough to be used in many applications. For example, they have been found to be insufficient for diagnosis of dental caries or periodontal pathosis, which are the most common diseases in clinical dentistry. Furthermore, increasing the resolution of such conventional methods requires increasing the intensity of applied x-rays, thus undesirably increasing the radiation dosage that patients are exposed to.

Accordingly, continuing efforts exist to improve the resolution of dental CBCT images.

### SUMMARY

The invention can be implemented in numerous ways. Accordingly, various embodiments of the invention are discussed below.

In one embodiment, a dental cone beam computed tomography (CBCT) system comprises: a photon generator configured to emit x-ray photons; a photon detector spaced apart from the photon generator so as to accommodate at least a portion of a human mouth therebetween, the photon detector configured to receive the x-ray photons; and a processor in electronic communication with the photon detector. The photon detector is a direct-conversion detector configured to convert each received x-ray photon directly to a corresponding electrical signal, to determine information corresponding to a spatial pattern of the electrical signals, and to transmit the information to the processor. The processor is further configured to generate an image of the portion of the human mouth from the transmitted information.

The photon generator and photon detector may be configured to face each other along a plurality of differing directions, so as to generate one or more of the images for each differing direction, each of the images being a two dimensional representation of the portion of the human mouth along its respective direction. The processor may be further configured to generate, from each of the generated two dimensional images, a three dimensional image of the portion of the human mouth.

The photon detector may further comprise a semiconductor layer in electrical communication with each of a plurality of pixels, the semiconductor layer configured to convert received ones of the photons to corresponding ones of the electrical signals. The pixels may be configured to generate the information according to individual received ones of the electrical signals.

The semiconductor layer may comprise an amorphous selenium layer. The amorphous selenium layer may have a thickness that is between 100 μm and 1500 μm.

The pixels may be arranged in an array having a pitch of 55 μm or less. The array may be a 256×256 array of the pixels.

In another embodiment, an x-ray imaging system comprises an assembly having an x-ray emitter positioned at one end thereof and an x-ray detector positioned at another end thereof, as well as a processor in electronic communication with the x-ray detector. The x-ray emitter and x-ray detector are positioned so as to accommodate one or more human teeth therebetween. The x-ray emitter is configured to emit x-ray photons through the one or more human teeth and toward the x-ray detector. The x-ray detector is a direct-conversion x-ray detector having an array of pixels each configured both to detect electrical signals corresponding to individual ones of the photons directed thereto, and to transmit a pixel signal to the processor, the pixel signal corresponding to the detected electrical signals. Also, the processor is configured to generate an image of the one or more human teeth from the collective pixel signals.

The assembly may be configured to rotate so as to place the x-ray emitter and x-ray detector at a plurality of differing positions, so as to generate one or more of the images at each differing position, each of the images being a two dimensional representation of at least a portion of the one or more human teeth. The processor may be further configured to generate, from each of the generated two dimensional images, a three dimensional image of the one or more human teeth.

The x-ray detector may further comprise a semiconductor layer in electrical communication with each of the pixels, the semiconductor layer configured to convert received ones of the photons to corresponding ones of the electrical signals. The pixels may each be configured to count corresponding individual received ones of the electrical signals, and to generate the corresponding pixel signal according to the count of electrical signals.

The array of pixels may be a 256×256 array of pixels. Each pixel may have a pitch of 55 μm or less.

In another embodiment, a dental cone beam computed tomography (CBCT) system comprises a photon generator configured to emit x-ray photons; a photon detector spaced apart from the photon generator so as to accommodate one or more human teeth therebetween, the photon detector configured to receive the x-ray photons; and a processor in electronic communication with the photon detector. The photon detector is further configured to generate a corresponding electrical signal from each received x-ray photon, to determine counts of individual ones of the electrical signals, and to transmit the counts to the processor. The processor is further configured to generate one or more images of the one or more human teeth from the collective counts.

The photon generator and photon detector may be configured to face each other along a plurality of differing directions, so as to generate one or more of the images for each differing direction, each of the images being a two dimensional representation of the one or more human teeth along its respective direction. The processor may be further configured to generate, from each of the generated two dimensional images, a three dimensional image of the one or more human teeth.

The photon detector may further comprise a semiconductor layer in electrical communication with each of a plurality of pixels, the semiconductor layer configured to convert received ones of the photons to corresponding ones of the electrical signals. The pixels may each be configured to generate a corresponding one of the counts as being a sum of the individual received ones of the electrical signals.

Other aspects and advantages of the invention will become apparent from the following detailed description taken in conjunction with the accompanying drawings which illustrate, by way of example, the principles of the invention.

### BRIEF DESCRIPTION OF THE DRAWINGS

For a better understanding of the invention, reference should be made to the following detailed description taken in conjunction with the accompanying drawings, in which:

FIG. 1 is a conceptual representation of a dental CBCT imaging apparatus constructed in accordance with embodiments of the invention;

FIG. 2 is a cutaway side view of a detector used in FIG. 1, illustrating further details of electronic components therein; and

FIGS. 3 and 4 are 3D and cutaway 2D images, respectively, illustrating views of a human tooth generated in accordance with embodiments of the invention.

Like reference numerals refer to corresponding parts throughout the drawings. The various Figures are not necessarily to scale.

### DETAILED DESCRIPTION OF EMBODIMENTS OF THE INVENTION

In one aspect, the invention relates to a dental CBCT apparatus that utilizes a novel x-ray detector having higher resolution. Unlike conventional CBCT x-ray detectors, the detectors of various embodiments of the invention are direct-conversion detectors that directly convert x-rays to electrical charges, and generate images according to a direct count of the detected charges. This is in contrast to conventional indirect detection, which does not directly convert x-rays to electrical charge, but rather converts x-rays to visible light, which is in turn converted to electrical charge by photodetectors to generate an image. Direct-conversion detection thus skips the step of converting x-rays to light, resulting in more accurate x-ray detection and thus higher image resolution, as well as images with less noise and greater contrast.

The direct-conversion detector of embodiments of the invention utilizes a semiconductor layer to capture incident x-rays and convert the x-rays to electrical charges, which are then detected to form images. This semiconductor layer can be, for example, an amorphous selenium layer.

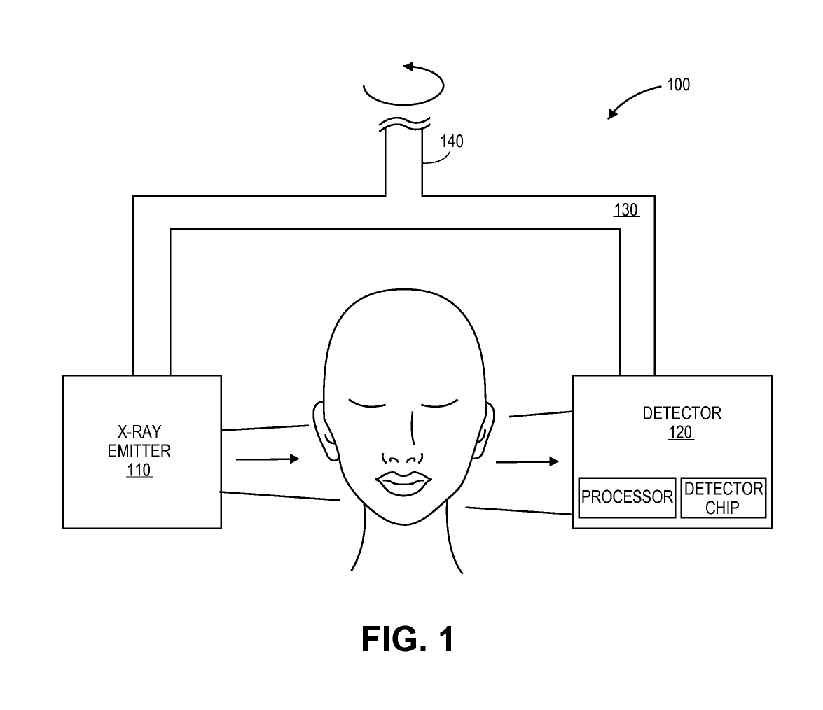

FIG. 1 is a conceptual representation of a direct-conversion dental CBCT imaging apparatus constructed in accordance with embodiments of the invention. Here, a CBCT system 100 includes an x-ray emitter 110 and an x-ray detector 120 that are connected by rotatable support 130. The support 130 maintains the x-ray emitter 110 and x-ray detector 120 a predetermined distance from each other, where the predetermined distance is one that is sized to allow one or more parts of a human body, such as a human head, to be positioned between the emitter 110 and detector 120, as shown. The detector 120 may contain a detector chip and a processor, as will be further described below. The x-ray emitter 110 can be a conventional x-ray source that emits a generally cone-shaped beam of x-ray particles or photons through the human patient's head (and in particular, at least part of his or her mouth) and onto the x-ray detector 120.

In this manner, one of ordinary skill in the art will observe that the CBCT system 100 can take a 2D x-ray image of the patient's mouth, and in particular his/her teeth. Furthermore, the rotatable support 130 is designed to pivot about its axis 140, which is generally aligned with the patient's mouth so that multiple x-ray images may be taken of the patient's teeth at different orientations. From these different 2D images, one of ordinary skill in the art will also observe that a 3D composite radiographic image may be constructed of one or more entire teeth.

A conventional digital x-ray detector would typically employ a matrix of photodetectors behind a phosphor screen or scintillator, and optical lens. In conventional operation, x-ray photons from an x-ray emitter would be directed into the phosphor screen, which converts incident x-rays to visible light. This light is then focused by the optical lens and projected onto the photodetectors, which act as a conventional camera capturing the image resulting from the generated visible light. Accordingly, indirect detectors such as these do not generate an image directly from its x-rays but rather indirectly generate an image from the visible light generated by the x-rays. However, as the generated visible light scatters or radiates away from the positions of its x-rays within the phosphor screen, a certain amount of blurring is inherent in any such image, resulting in reduced resolution. Accordingly, there are inherent limits to the resolution of any indirect x-ray detector.

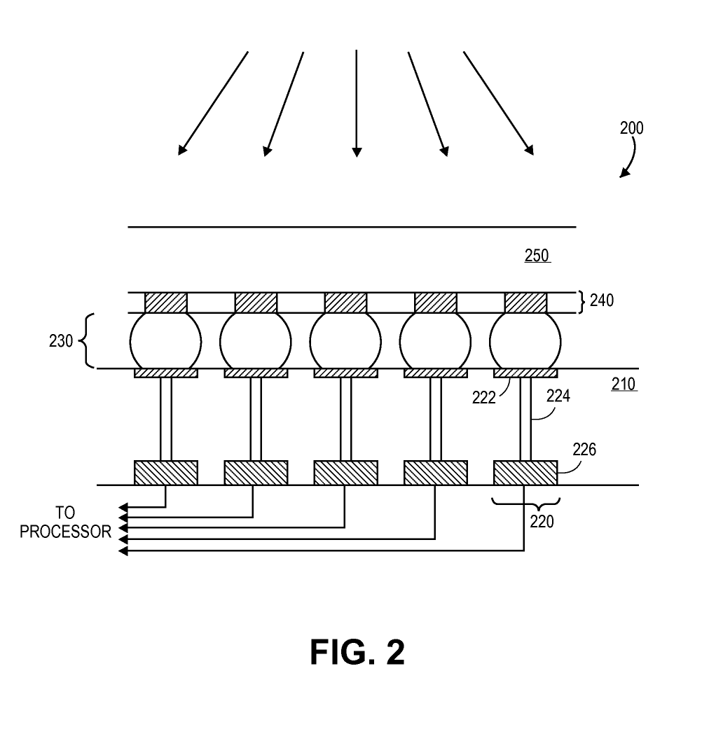

In contrast, x-ray detector 120 is a direct-conversion detector designed to directly convert incident x-ray photons to corresponding electrical charges, rather than first converting them to visible light and then converting that visible light to electrical charge. Thus, it does not contain a scintillator or photodetectors, but instead utilizes a semiconductor layer that directly converts x-rays to electrical charge. FIG. 2 is a cutaway side view of one such detector. Here, x-ray detector 120 employs a pixel readout chip bump bonded to an x-ray detection chip. More specifically, an integrated circuit assembly 200 has a pixel readout chip 210 with a matrix of pixel cells 220. A number of solder bumps 230 electrically connect each pixel cell 220 to electrical leads 240, and a semiconductor layer 250 is deposited over the leads 240 to be electrically connected thereto.

Each pixel cell 220 produces an individual pixel of an image, and includes an electrical contact 222 making contact with a solder bump 230, electrical connector 224, and pixel circuitry 226. Each vertically aligned set comprising an electrical lead 240, solder bump 230, contact 222 and connector 224 collectively provides an electrical pathway between adjacent portions of semiconductor layer 250 and the pixel circuitry 226, allowing pixel circuitry 226 to detect electrical currents generated by x-ray photons that fall incident to that region of semiconductor layer 250. In this manner, the readouts of pixel cells 220 collectively describe the spatial pattern of electrical signals generated by incident x-rays, which information can be used to generate an image of material that the x-rays have passed through.

Each instance of pixel circuitry 226 shown is a block representation of any set of circuitry that can operate to count electrical signals generated by individual x-ray photons in semiconductor layer 250, and emit a readout signal corresponding to the count. Such circuitry is known.

In operation, x-ray photons emitted by x-ray emitter 110 (represented by the arrows in the upper portion of FIG. 2) are directed toward the x-ray detector 120, where they first pass through the patient's tissues and then enter the semiconductor layer 250. There, they are converted to electrical signals that propagate through semiconductor layer 250 to nearby leads 240. These signals are then transmitted to the corresponding pixel circuit 226, which registers each detected signal as corresponding to a single received x-ray photon. In more detail, the pixel circuit 226 includes a counter that counts the electrical signals it receives, each signal corresponding to a single x-ray photon.

The pixel circuit 226 can include any known circuitry for counting or otherwise accumulating signals, and outputting this count or accumulation in order to form an image. Such circuitry can employ any type and number of modes or methods for detecting the electrical signals from x-ray photons generally, or for counting photons specifically. For example, in one mode, it may simply accumulate or sum the total charge detected at each pixel. Alternatively, it may count the number of signals whose energy exceeds a predetermined threshold energy value, where this threshold value can be any suitable value. Other embodiments contemplate modes in which the circuitry counts the number of signals that exceed an energy threshold for at least a minimum time (e.g., a time over threshold mode), or counts the number of signals that exceed an energy threshold within a certain maximum time (e.g., a time of arrival mode). Any type and combination of modes is contemplated. The pixel circuits 226 can include amplifier, energy discriminator, counter, and other circuits for implementing these and other modes.

If the detector 120 counts individual photons, i.e. individual charges from x-ray photons, the counts from each pixel circuit 226 are transmitted to a processor, which may be a separate integrated circuit within x-ray detector 120, or may be remotely located, i.e. outside of x-ray detector 120. The processor assigns a visual indicator (e.g., a brightness or color value) to the count value for each pixel, thus assembling a 2D image from the individual pixel values. The CBCT system 100 may then be rotated about axis 140 and another 2D image may be taken as above. By repeating this process at different rotational positions, a number of 2D images may be generated. The processor can then use the information in each 2D image to generate a corresponding 3D image of the tooth or other structure that has been scanned.

The detector 120 may also generate each 2D image in different ways, such as by summing the amount of charge detected at each pixel, rather than counting individual charges. Different visual indicators would then be assigned to different summed charge levels, e.g. brightness values would be a function of total summed charge detected over some period of time, such as the duration of the x-ray pulse emitted by emitter 110.

One of ordinary skill in the art will realize that the integrated circuit assembly 200 can contain any number and arrangement of pixels. That is, any number and arrangement of pixel circuits 226 is contemplated, along with their corresponding structures 222, 224, 230, 240. In one exemplary and nonlimiting configuration, the integrated circuit 200 can contain a 256×256 array of pixels arranged in a square matrix format, with a pitch (i.e., pixel size, corresponding to resolution) of 55 μm. This particular configuration can be found in, for example, the Timepix and Medipix application specific integrated circuits (ASICs) produced and sold by X-ray Imaging Europe GmbH. Such 55 μm resolution is a significant improvement over current CBCT image resolution.

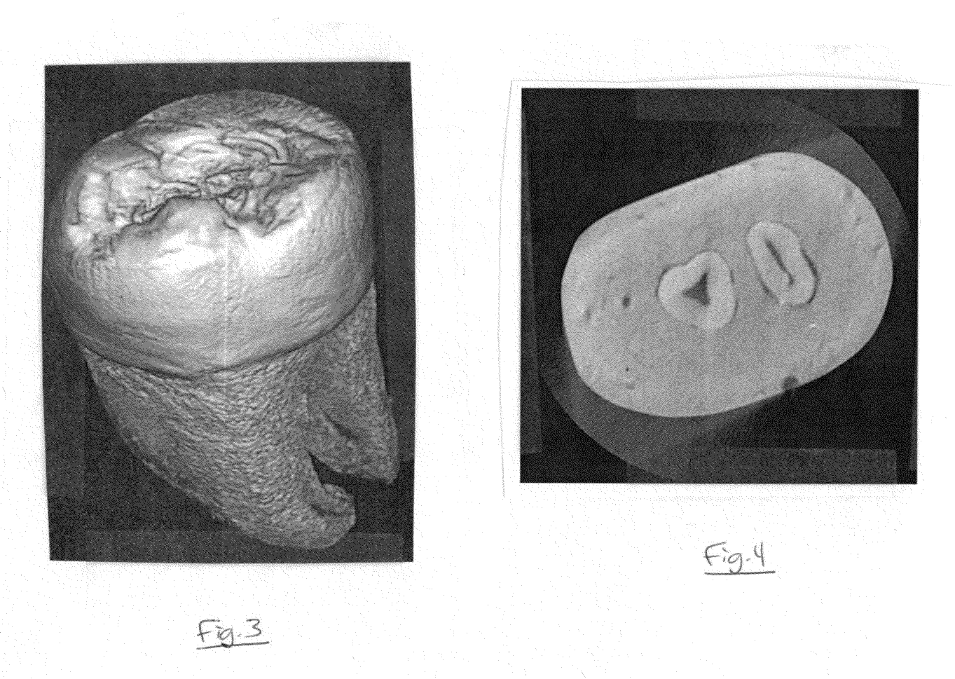

One of ordinary skill in the art will also realize that the semiconductor layer 250 can be any semiconductor material that can convert incident x-ray photons or particles to electrical signals. Exemplary semiconductive materials can include silicon, selenium, cadmium telluride, cadmium zinc telluride, and the like. It has been found that one such suitable material is amorphous selenium (a-Se). In particular, an a-Se layer of 100-1500 μm thickness is suitable for captures of images of hard tissue (e.g., human teeth and bones), although any semiconductor layer thickness can be employed. Embodiments of the present invention were implemented and tested to determine their resolution. FIGS. 3 and 4 are 3D and cutaway 2D images, respectively, illustrating views of human teeth generated in accordance with embodiments of the invention. FIG. 3 is a 3D reconstruction of a tooth, which was produced using 200 2D projections at angles between 0 and 180° of rotation. The 2D projections were made with a Timepix detector ASIC using a 300 μm thick silicon semiconductor layer. From FIG. 3, it can be seen that the methods and apparatuses of embodiments of the invention are capable of producing 3D dental images of superior resolution to conventional images.

FIG. 4 is a sectional image of a sample of two extracted human molars invested in plaster. The sample was placed between an x-ray source (50 kVp, 5 mA, 0.6 s) and an x-ray detector with a 200 μm thick a-Se semiconductor layer and an 85 μm pixel pitch. The sample, source, and detector were mounted on a mechanical rotation table, and 250 basis images were taken per 1°, using manual rotation. From FIG. 4, it can be seen that the methods and apparatuses of embodiments of the invention are capable of producing 2D dental images of superior resolution to conventional images.

Embodiments of the invention provide a number of significant advantages over conventional CBCT devices. As above, the increased resolution (e.g., 55 μm) allows for diagnoses that previously could not be made solely with CBCT images. For instance, dental caries and periodontal pathosis can be accurately diagnosed from images such as FIG. 3.

As another example, the need for dental impressions is reduced or eliminated. A dental impression is an imprint of hard tissue as well as soft tissue, and can be generated with specific types of impression materials depending on the specific application, such as Prosthodontics, Maxillofacial prosthetics, Restorative, Orthodontics, diagnosis and Oral and Maxillofacial surgery. The material for an impression can vary depending on the application. However, the purpose of taking impressions is to capture part or all of a person's dentition and surrounding oral cavity structures as correctly as clinically needed. The dental impression forms a negative mold of hard and soft tissues, which can then be used to make a cast or a model of the given anatomy. Casts are used for diagnostics, patient record, treatment planning, fabrication of custom trays, fabrication of dentures, crowns or other prostheses and orthodontics. However, casting the imprints is a slow, uncomfortable, and laborious technique requiring patients to make multiple visits in their dentists' office. Generation of imprints using digital cone-beam CT images would be much quicker. For instance, the 3D models could be sent to the laboratory electronically.

Additionally, such images are generally more accurate than physical impressions, leading to fewer bad castings. Castings can be made from 3D models by, for example, recording 2D images as above, where the images are in a standard format such as DICOM formatted files, converting the DICOM files to .stl files or files of any other desired format, and 3D printing or machining a casting using the .stl or other formatted files to provide the position information for the 3D casting.

The foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description. They are not intended to be exhaustive or to limit the invention to the precise forms disclosed. Many modifications and variations are possible in view of the above teachings. For example, embodiments of the invention contemplate use of any detector besides a Timepix or Medipix detector, so long as sufficient resolution is provided to allow for dimensionally accurate 3D models that can be used as clinically valuable diagnostic tools. Also, embodiments of the invention contemplate use of a-Se or any other suitable material in the detector, so long as sufficient resolution is provided to allow for dental images with improved resolution. The detector can employ an a-Se layer of any thickness. Embodiments of the invention contemplate direct-conversion detection of photons or particles (x-ray or otherwise) in any manner, such as by summing and/or counting charges. All numerical values are approximate, and may vary. The embodiments were chosen and described in order to best explain the principles of the invention and its practical applications, to thereby enable others skilled in the art to best utilize the invention and various embodiments with various modifications as are suited to the particular use contemplated. Any one or more of the various described features of any embodiments of the invention may be mixed and matched in any manner, to form further embodiments also within the scope of the invention.

1. A dental cone beam computed tomography (CBCT) system, comprising:
a photon generator configured to emit x-ray photons;
a photon detector spaced apart from the photon generator so as to accommodate at least a portion of a human mouth therebetween, the photon detector configured to receive the x-ray photons; and
a processor in electronic communication with the photon detector;
wherein the photon detector is a direct-conversion detector configured to convert each received x-ray photon directly to a corresponding electrical signal, to determine information corresponding to a spatial pattern of the electrical signals, and to transmit the information to the processor; and
wherein the processor is further configured to generate an image of the portion of the human mouth from the transmitted information.

2. The CBCT system of claim 1, wherein:
the photon generator and photon detector are configured to face each other along a plurality of differing directions, so as to generate one or more of the images for each differing direction, each of the images being a two dimensional representation of the portion of the human mouth along its respective direction; and
the processor is further configured to generate, from each of the generated two dimensional images, a three dimensional image of the portion of the human mouth.

3. The CBCT system of claim 1, wherein:
the photon detector further comprises a semiconductor layer in electrical communication with each of a plurality of pixels, the semiconductor layer configured to convert received ones of the photons to corresponding ones of the electrical signals; and
the pixels are configured to generate the information according to individual received ones of the electrical signals.

4. The CBCT system of claim 3, wherein the semiconductor layer comprises an amorphous selenium layer.

5. The CBCT system of claim 4, wherein the amorphous selenium layer has a thickness that is between 100 μm and 1500 μm.

6. The CBCT system of claim 3, wherein the pixels are arranged in an array having a pitch of 55 μm or less.

7. The CBCT system of claim 6, wherein the array is a 256×256 array of the pixels.

8. An x-ray imaging system, comprising:
an assembly having an x-ray emitter positioned at one end thereof and an x-ray detector positioned at another end thereof, the x-ray emitter and x-ray detector further being positioned so as to accommodate one or more human teeth therebetween; and
a processor in electronic communication with the x-ray detector;
wherein the x-ray emitter is configured to emit x-ray photons through the one or more human teeth and toward the x-ray detector;
wherein the x-ray detector is a direct-conversion x-ray detector having an array of pixels each configured both to detect electrical signals corresponding to individual ones of the photons directed thereto, and to transmit a pixel signal to the processor, the pixel signal corresponding to the detected electrical signals; and
wherein the processor is configured to generate an image of the one or more human teeth from the collective pixel signals.

9. The x-ray imaging system of claim 8, wherein:
the assembly is configured to rotate so as to place the x-ray emitter and x-ray detector at a plurality of differing positions, so as to generate one or more of the images at each differing position, each of the images being a two dimensional representation of at least a portion of the one or more human teeth; and
the processor is further configured to generate, from each of the generated two dimensional images, a three dimensional image of the one or more human teeth.

10. The x-ray imaging system of claim 8, wherein:
the x-ray detector further comprises a semiconductor layer in electrical communication with each of the pixels, the semiconductor layer configured to convert received ones of the photons to corresponding ones of the electrical signals; and
the pixels are each configured to count corresponding individual received ones of the electrical signals, and to generate the corresponding pixel signal according to the count of electrical signals.

11. The x-ray imaging system of claim 10, wherein the semiconductor layer comprises an amorphous selenium layer.

12. The x-ray imaging system of claim 11, wherein the amorphous selenium layer has a thickness that is between 100 μm and 1500 μm.

13. The x-ray imaging system of claim 8, wherein the array of pixels comprises a 256×256 array of pixels having a pitch of 55 μm or less.

14. A dental cone beam computed tomography (CBCT) system, comprising:
a photon generator configured to emit x-ray photons;
a photon detector spaced apart from the photon generator so as to accommodate one or more human teeth therebetween, the photon detector configured to receive the x-ray photons; and
a processor in electronic communication with the photon detector;
wherein the photon detector is further configured to generate a corresponding electrical signal from each received x-ray photon, to determine counts of individual ones of the electrical signals, and to transmit the counts to the processor; and
wherein the processor is further configured to generate one or more images of the one or more human teeth from the collective counts.

15. The CBCT system of claim 14, wherein:
the photon generator and photon detector are configured to face each other along a plurality of differing directions, so as to generate one or more of the images for each differing direction, each of the images being a two dimensional representation of the one or more human teeth along its respective direction; and
the processor is further configured to generate, from each of the generated two dimensional images, a three dimensional image of the one or more human teeth.

16. The CBCT system of claim 14, wherein:
the photon detector further comprises a semiconductor layer in electrical communication with each of a plurality of pixels, the semiconductor layer configured to convert received ones of the photons to corresponding ones of the electrical signals; and
the pixels are each configured to generate a corresponding one of the counts as being a sum of the individual received ones of the electrical signals.

17. The CBCT system of claim 16, wherein the semiconductor layer comprises an amorphous selenium layer.

18. The CBCT system of claim 17, wherein the amorphous selenium layer has a thickness that is between 100 μm and 1500 μm.

19. The CBCT system of claim 16, wherein the pixels are arranged in an array having a pitch of 55 μm or less.

20. The CBCT system of claim 19, wherein the array is a 256×256 array of the pixels.
